(19) United States
(12) Patent Application Publication
(10) Pub. No.: US 2026/0028592 A1
(43) Pub. Date: Jan. 29, 2026

(54) TGFB TREATMENT OF ORGANOID CULTURES TO PRIME FOR REGENERATIVE THERAPY OF DAMAGED GI EPITHELIA

(71) Applicant: RUTGERS, THE STATE UNIVERSITY OF NEW JERSEY, New Brunswick, NJ (US)

(72) Inventors: Lei CHEN, New Brunswick, NJ (US); Michael P. VERZI, New Brunswick, NJ (US)

(73) Assignee: RUTGERS, THE STATE UNIVERSITY OF NEW JERSEY, New Brunswick, NJ (US)

(21) Appl. No.: 18/997,744

(22) PCT Filed: Jul. 25, 2023
(86) PCT No.: PCT/US2023/028568

§ 371 Date: Jan. 22, 2025

Related U.S. Application Data
(60) Provisional application No. 63/392,365, filed on Jul. 26, 2022.

(51) Int. Cl. C12N 5/071; A61L 27/36; C12N 5/0775

(52) U.S. Cl. CPC C12N 5/068; A61L 27/3629; C12N 5/0662; A61L 2430/22; C12N 2501/15; C12N 2510/00; C12N 2513/00

(57) ABSTRACT

Certain emboidments provide a method of promoting regeneration of one or more intestinal stem cells in a mammalian organoid culture, the method comprising treating the organoid culture with an effective amount of transforming growth factor beta (TGFB). Certain embodiments also provide a method for preparing an organoid culture comprising intestinal stem cells and a method of treating a subject with damaged or defective intestinal tissue comprising engrafting an organoid into the subject.

### REFERENCE TO RELATED APPLICATIONS

This application claims priority to U.S. Provisional Application No. 63/392,365 that was filed on Jul. 26, 2022. The entire content of the applications referenced above is hereby incorporated by reference herein.

### BACKGROUND

Radiotherapy, chemotherapy, inflammatory bowel disease, graft versus host disease, and other maladies of the gastrointestinal (GI) tract can all induce extensive damage of the intestinal epithelial lining. These conditions could be alleviated by transplantation of healthy epithelial cells. Genetic disorders affecting intestinal epithelial cell functions, such as microvillus inclusion disorders, congenital diarrhea, and nutrient transporter deficiencies, could also be corrected by replacement of defective epithelium with genetically corrected epithelial transplants. Epithelial transplants have been demonstrated in animal models, but they require optimization to accelerate use in the clinical setting, including improved methods for promoting tissue engraftment and for reducing the incidence and extent of transplant rejection. Accordingly, improved methods for transplantation of tissues, e.g., into the GI tract, are needed.

### BRIEF SUMMARY

Damage caused by radiotherapy, chemotherapy, or acute inflamation is most detrimental in the crypt region of the intestinal epithelium, where stem cells and progenitor cells reside. Immune cells and mesenchymal cells cooperate to promote epithelial regeneration in the intestine after injury (Hageman et al., Developmental Cell 2020). As described herein, it was hypothesized that macrophage-derived TGFB promotes intestinal epithelial regeneration by inducing cells into a regenerative state. Herein it is shown that TGFB1 plays a role in promoting intestinal regeneration. A novel application of these findings to enhance intestinal engraftment by using TGFB1 ligands to induce a regenerative state in intestinal organoids is described. It is demonstrated that TGFB1-treated organoids support more robust tissue engraftment in a mouse model of ulcerative colitis. Further, it is shown that treatment of adult human duodenal organoid cultures with TGFB1 induced a similar set of regenerative genes to those observed in the mouse model, indicating that these results can be translated into humans.

Accordingly, certain embodiments provide a method of promoting regeneration of one or more intestinal stem cells in a mammalian organoid culture, the method comprising treating the organoid culture with an amount of transforming growth factor beta (TGFB) effective to promote intestinal epithelial regeneration.

In certain embodiments, the TGFB comprises transforming growth factor beta-1 (TGFB1).

In certain embodiments, the organoid culture comprises epithelial regenerative cells.

In certain embodiments, the epithelial regenerative cells comprise at least one of a Clusterin (Clu) and Stem cell antigen-1 (Ly6a) biomarker.

In certain embodiments, the organoid cultures compreses epithelial stem cells.

In certain embodiments, the TGFB increases stemness of intestinal epithelial cells.

In certain embodiments, the organoid culture comprises mesenchymal cells or mesenchymal stem cells.

In certain embodiments, the TGFB increases stemness of intestinal mesenchymal cells.

In certain embodiments, the organoid culture comprises cells that express a TGFB1 receptor.

In certain embodiments, the TGFB1 receptor is transforming growth factor beta receptor 2 (Tgfbr2).

In certain embodiments, the organoid culture comprises a spherical morphology.

In certain embodiments, the organoid culture expresses increased levels of fetal or regenerative biomarkers as compared to an untreated control organoid culture.

In certain embodiments, the regenerative biomarkers include at least one of Clu and Connective tissue growth factor (Ctgf) biomarkers.

In certain embodiments, the organoid culture comprises cells that express at least one of Lgr5 and Olfm4 biomarkers.

Certain embodiments provide a method of treating a subject with damaged or defective intestinal tissue comprising engrafting an organoid into the intestine of the subject, wherein the organoid has been pre-treated with an amount of TGFB effective to promote intestinal epithelial regeneration.

In certain embodiments, the TGFB comprises TGFB1.

In certain embodiments, the organoid comprises epithelial regenerative cells.

In certain embodiments, the epithelial regenerative cells comprise at least one of a Clu and Ly6a biomarker.

In certain embodiments, the the organoid comprises epithelial stem cells.

In certain embodiments, the TGFB increases stemness of intestinal epithelial cells.

In certain embodiments, the organoid comprises mesenchymal cells or mesenchymal stem cells.

In certain embodiments, the TGFB increases the stemness of intestinal mesenchymal cells.

In certain embodiments, the organoid comprises cells that express a TGBF1 receptor.

In certain embodiments, the TGFB1 receptor is Tgfbr2.

In certain embodiments, the organoid comprises a spherical morphology.

In certain embodiments, the organoid expresses increased levels of fetal or regenerative biomarkers as compared to an untreated control organoid.

In certain embodiments, the regenerative biomarkers include at least one of Clu and Ctgf biomarkers.

In certain embodiments, the organoid comprises cells that express at least one of Lgr5 and Olfm4 biomarkers.

In certain embodiments, the treated intestinal tissue comprises an increased number of organoid colonies in comparison to intestinal tissue engrafted with an organoid that has not been pre-treated with TGFB.

In certain embodiments, the treated intestinal tissue comprises an increased size of organoid colonies in comparison to intestinal tissue engrafted with an organoid that has not been pre-treated with TGFB.

In certain embodiments, the treated intestinal tissue comprises an increased area of organoid colonies in comparison to intestinal tissue engrafted with an organoid that has not been pre-treated with TGFB.

In certain embodiments, the subject was previously treated with at least one of chemotherapy and radiotherapy.

In certain embodiments, the damaged or defective intestinal tissue is caused by at least one of the following diseases or disorders: inflammatory bowel disease, graft versus host disease, pathogens and other diseases or disorders causing ulceration of intestinal tissue.

In certain embodiments, the damaged or defective intestinal tissue is caused by a genetic disease or disorder affecting intestinal epithelial cell functions.

In certain embodiments, the genetic disease or disorder comprises at least one of the following: microvillus inclusion disorders, congenital diarrhea, and nutrient transporter deficiencies.

In certain embodiments, the organoid comprises a genetically corrected organoid.

Other objects, features, and advantages of the present invention will be apparent to one of skill in the art from the following detailed description and figures.

### BRIEF DESCRIPTION OF THE FIGURES

FIG. 1. Demonstration of intestinal regeneration following 12 Gy irradiation of mice. OLFM4 and Ki67 immunostains are used to identify stem and proliferative cells, respectively.

FIG. 2. Analysis of scRNAseq data following mouse irradiation at days 0, 1, 3, 7, and 14. UMAP projection shows diversity of cell populations captured and the representation of cells in these clusters over the timecourse. Dot plot indicates that among secreted regulators of the TGF/BMP/WNT signaling pathways, transcripts corresponding to TGFB1 are the most upregulated during regeneration of the gut (dashed box) and overlap expression of epithelial regenerative markers Ly6a and Clu. Public scRNAseq data obtained from GSE165318 (GEO accession number).

FIG. 3. UMAP projection of all cells identifies a cell cluster expressing highest levels of Tgfb1. Levels are most enriched at day 3 post-irradiation.

FIG. 4. The same UMAP projection of cells is used to demonstrate that the Tgfb1-expressing cells co-express markers of monocyte/macrophages.

FIGS. 5A-5B. Tissues from mice at different times following 12 Gy irradiation were probed for the monocyte/macrophage marker F4/80 using immunohistochemistry and reveal an increase in this cell population as the tissue begins to heal at 2 days post-IR. (FIG. 5A) qRT-PCR analysis indicates that transcripts of monocyte/macrophage marker genes are also elevated in intestines at 3 days post-IR. (FIG. 5B)

FIG. 6. RNAscope was used to localize Tgfb1 transcripts with immunostaining signal from ECAD and F4/80. These co-stains reveal that F4/80-marked macrophages are associated with damaged crypt epithelium at day 3 post-IR, and that they overlap with regions of elevated levels of Tgfb1, suggesting that monocyte/macrophages are recruited to the damaged tissue and produce TGFB1.

FIG. 7. Monocyte/Macrophages were depleted using clodronate-containing liposomes (2 treatments of 200 ul IP injections 72 hours pre- and day of irradiation) and immunostaining was performed on control or treated mice to confirm reduction in F4/80 expressing cells upon treatment. Tissues were collected 3 days post-irradiation.

FIGS. 8A-8B. Tissues from the same experiment performed in Slide 7 were assessed for their state of regeneration using OLFM4 immunostain. (FIG. 8A) Clodronate-treated samples had a significant reduction in the number of OLFM4+ regenerating cell clusters. (FIG. 8B)

FIGS. 9A-9B. Mice treated with 2 doses of neutralizing antibodies directed against TGFB were less efficient at regenerating post irradiation compared to control-treated mice, as marked by OLFM4 immunostaining (FIG. 9A) and as measured by counting the number of proliferative foci. (FIG. 9B)

FIG. 10. Tgfbr2 intestine-specific knockout restricts regeneration after irradiation. Mice were treated with tamoxifen to inactivate Tgfbr2 in the intestinal epithelium 7 days before 12 gy irradiation. Three days post-IR, intestine was collected and scored for regenerative foci using OLFM4 immunostaining.

FIG. 11. Smad4 intestine-specific knockout restricts regeneration after irradiation. Mice were treated with tamoxifen to inactivate Smad4 in the intestinal epithelium 7 days before 12 gy irradiation. Three days post-IR, intestine was collected and score for regenerative foci using OLFM4 immunostaining.

FIG. 12. scRNAseq of mice intestine across a timecourse post-irradiation. Epithelial cells (marked by Epcam expression) were further investigated for expression of regeneration markers (Clu and Ly6a) and Tgfbr2. Of all the epithelial cells in the dataset, there is a strong correlation between Tgfbr2 and the subset of cells expressing regenerative markers.

FIG. 13A. 24-hour treatment of wild-type mouse organoids with TGFB1 induces a robust regenerative/fetal spheroid gene signature. Bulk RNA-seq of intestinal organoids cultured either in the presence or absence of TGFB1 show strong correlation with published gene signatures associated with intestinal regeneration post-DSS injury or fetal enteroids, as measured by GSEA analysis. For published gene signatures see Yui S, et al., YAP/TAZ-Dependent Reprogramming of Colonic Epithelium Links ECM Remodeling to Tissue Regeneration, Cell Stem Cell. 2018 Jan. 4;22(1):35-49.e7; Mustata RC, et al., Identification of Lgr5-independent spheroid-generating progenitors of the mouse fetal intestinal epithelium, Cell Rep. 2013 Oct. 31;5(2):421-32.

FIG. 13B. qRT-PCR analysis of intestinal organoids cultured either in the presence or absence of a 24 hr treatment with 2 ng/ml TGFB1 show elevation of regenerative cell marker transcripts. Elevation in response to TGFB1 treatment is dependent upon Tgfbr2 expression, as organoids genetically engineered to lack the receptor do not increase regeneration gene expression in response to TGFB1 treatment.

FIG. 14. 24-hour treatment of wild-type mouse organoids with TGFB1 induces a robust revival stem cell and YAP gene signature. Bulk RNA-seq of intestinal organoids cultured either in the presence or absence of TGFB1 show strong correlation with published gene signatures associated with intestinal regeneration post-DSS injury or fetal enteroids, as measured by GSEA analysis. For published gene signatures see Ayyaz A. et al., Single-cell transcriptomes of the regenerating instestine reveal a revival stem cell, Nature 2019 May 569(7754):121-125; Gregorieff A, et al., Yap-dependent reprogramming of Lgr5⁺ stem cells drives intestinal regeneration and cancer, Nature 2015 October 526:715-518.

FIGS. 15A-15B. Organoids treated with TGFB1 acquire a spheroid morphology (FIG. 15A) and maintain expression of regeneration marker genes for at least 5 days post-TGFB1 treatment (FIG. 15B).

FIG. 16. Schematic of experimental design for data depicted in FIGS. 17-20.

FIG. 17. scRNAseq of organoids post-irradiation and TGFB1-treatment, as depicted in FIG. 16. UMAP plots show that across timepoints, there is a close correlation between Clu and Tgfbr2 expression.

FIG. 18. scRNAseq of organoids post-irradiation and TGFB1-treatment, as depicted in FIG. 16. UMAP plots show that across timepoints, Lgr5 and Olfm4-expressing clusters begin to overlap with Clu-expressing cells.

FIG. 19. scRNAseq of organoids post-irradiation and TGFB1-treatment, as depicted in FIG. 16. RNA velocity analysis identifies cells in Lgr5 and Olfm4-expressing clusters are synthesizing new Clu transcripts.

FIGS. 20A-20B. Experimental design for organoid transplantation assay, to determine the ability of TGFB1 to prime organoids to engraft into damaged colonic tissue. Competition assay illustrates repair of intestinal injury in TGFB1-treated vs vehicle-treated organoids for transplantation. There is a focus on the priming of organoids prior to transplantation. (FIG. 20A) Micrographs illustrating the transgenic organoid lines used to visualize transplants, treated with either TGFB1 to induce regenerative spheres, or vehicle-treated controls. (FIG. 20B)

FIG. 21. Continued experimental design for organoid transplantation assay, with focus on the transfer into DSS-treated immunodeficient mice and data collection.

FIG. 22. Fluorescent micrographs demonstrating transgenic organoid grafts into mice, as depicted in the experimental schematics of FIGS. 20-21.

FIGS. 23A-23B. Quantification of transplant efficiency resulting from the experimental design on slides 20-21. Depiction of the average size of organoid grafts per colonized area (FIG. 23A) and average size of organoid grafts per mouse (FIG. 23B) observed. Symbol type represents a single mouse used in the competition assay.

FIG. 24. Treatment of adult human duodenal organoids with TGFB1 elicits a similar response to mouse organoids, specifically, the induction of genes associated with intestinal regeneration.

FIG. 25. Mesenchymal cells isolated from control or irradiated mice (3 days post-IR, 12 Gy) were better able to induce a regeneration gene expression profile when co-cultured with intestinal organoids.

FIG. 26. Co-culture of organoids with irradiated mesenchyme elicits an upregulation of genes associated with regeneration of the intestinal epithelium (see FIG. 25), and this upregulation is dependent upon TGFB-signaling, as treatment with TGFB receptor inhibitors SB525334 or A83-01 lead to suppression of transcripts associated with regenerating intestinal epithelium.

### DETAILED DESCRIPTION

It was hypothesized that macrophage-derived Transforming Growth Factor-beta (TGFB) promotes intestinal epithelial regeneration by inducing cells into a regenerative state. As described herein, this hypothesis was tested by treating intestinal organoid cultures with Transforming Growth Factor-beta-1 (TGFB1 or Tgfb1) and monitoring gene expression changes. See, e.g., Moses et al., The Discovery and Early Days of TGF-β: A Historical Perspective, Cold Spring Harb. Perspect. Biol. 8, a021865 (2016). Gene expression analysis revealed that organoids treated with TGFB1 underwent a dramatic shift in gene expression profile, upregulating transcripts associated with regenerative epithelium and TGFB1 signaling. These gene expression changes in response to TGFB1 treatment were shown to be dependent upon the organoids expressing the receptor for TGFB1 ligand, Transforming Growth Factor Beta Receptor 2 (TGFBR2 or Tgfbr2). Moreover, competition assays were performed to test the ability of control-treated and TGFB1-treated organoids to engraft into damaged intestines of mice. The results of these assays demonstrated a clear advantage for the TGFB1-treated organoids to engraft into mouse intestines compared to control-treated organoids, with both increased numbers of organoid colonies forming and increased engraftment areas in the recipient colon tissues. This data supports the notion that TGFB1 increases the stemness of intestinal organoids by inducing intestinal regeneration and helping to restore stem cells. As such, pre-treatment of GI tract transplants using TGFB represents a method to increase regeneration of tissue and transplant efficacy.

As used herein, the following terms have the meanings ascribed to them unless specified otherwise.

As used herein, an “antibody” is any immunoglobulin polypeptide, or fragment thereof, having immunogen binding ability.

As used herein, an “agonist” is an agent that causes an increase in the expression or activity of a target gene or protein, respectively. An agonist can bind to and activate its cognate receptor in some fashion, which directly or indirectly brings about this physiological effect on the target gene or protein.

As used herein, an “inhibitor” is an agent that causes a decrease in the expression or activity of a target gene or protein, respectively. An “antagonist” can be an inhibitor, but is more specifically an agent that binds to a receptor, and which in turn decreases or eliminates binding by other molecules.

As used herein, “intestinal epithelium,” “intestinal epithelial cells,” “intestinal mesenchyme,” “intestinal mesenchymal cells” or “intestinal tissue” refers to the portion of the mammalian alimentary canal that extends from the stomach to the rectum, including the small intestine, the large intestine, and the colon. In some embodiments, mammalian intestinal tissue comprises mouse intestinal tissue. In some embodiments, mammalian intestinal tissue comprises human intestinal tissue.

As used herein, the term “stem cell” refers to a multipotent cell having the capacity to self-renew and to differentiate into multiple cell lineages.

As used herein, the term “stemness” refers to the capability of cells for self-renewal, regeneration, and/or differentiation.

As used herein, the term “epithelial stem cell” refers to a multipotent cell which has the potential to become committed to multiple cell lineages, including cell lineages resulting in epithelial cells.

As used herein, the term “mesenchymal stem cell” refers to a multipotent cell which has the potential to become committed to multiple cell lineages, including cell lineages resulting in mesenchymal cells.

As used herein, the term “progenitor cell” refers to a lineage-restricted cell derived from a stem cell.

As used herein, the term “engraft” or “engraftment” refers to the process of stem or progenitor cell incorporation into a tissue of interest in vivo through contact with existing cells of the tissue.

As used herein, a “population” of cells is any number of cells greater than 1, but is preferably at least 1×10² cells, at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, the term “organoid” or “epithelial organoid” refers to a cell cluster or aggregate that resembles an organ, or part of an organ, and possesses cell types relevant to that particular organ.

As used herein, the terms “culture,” “cell culture,” “tissue culture,” and “organoid culture” are used to refer to a system capable of maintaining, facilitating, and/or enhancing the growth, function, and/or viability of a cell, tissue, or organoid.

As used herein, a “subject” is a vertebrate, including any member of the class mammalia.

As used herein, a “mammal” refers to any mammal including but not limited to human, mouse, rat, sheep, monkey, goat, rabbit, hamster, horse, cow or pig.

As used herein, “expression” refers to the transcription and/or translation of an endogenous gene, heterologous gene or nucleic acid segment, or a transgene in cells. Expression may also refer to the production of protein.

As used herein, “gene” refers to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins.

As used herein, “increasing” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100% or more, for example, as compared to the level of a reference.

As used herein, “increases” also means increases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a as compared to the level of a reference standard.

As used herein, “decreasing” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference.

As used herein, “decreases” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference.

As used herein, the term “control” means a standard or reference condition (e.g., untreated with a test agent or combination of test agents).

As used herein, the term “canonical” or “canonically” refers to generalized, common, or standard features, pathways, or treatments.

As used herein, “promoting” and derivations thereof, may be used interchangeably with terms such as “activate,” “enhance,” “stimulate,” “increase,” and like terms, as appropriate in the context. It will be understood that the magnitude or extent of promotion, stimulation, activation, enhancement, or an increase described herein may vary.

For example, promoting proliferation, regeneration, and/or self-renewal of one or more intestinal stem cells and/or non-stem intestinal progenitor cells may refer to an increase in proliferation, regeneration, and/or self-renewal of one or more intestinal stem cells and/or non-stem intestinal progenitor cells by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% relative to level of proliferation, regeneration, and/or self-renewal in the absence of an agent or composition described herein. In some embodiments, stimulating the proliferation, regeneration, and/or self-renewal of one or more intestinal stem cells and/or non-stem intestinal progenitor cells increases the proliferation and/or self-renewal of such cells by at least 1.1 fold, 1.2 fold, 1.3 fold, 1.4 fold, 1.5 fold, 1.6 fold, 1.7 fold, 1.8 fold, 1.9 fold, 2.0 fold, 2.5 fold, 3 fold, 4 fold, 5 fold, 6 fold, 7 fold, 8 fold, 9 fold, 10 fold, 20 fold, 25 fold, 33 fold, 40 fold, 50 fold, 60 fold, 70 fold, 80 fold, 90 fold, or 100 fold or more.

As another example, promoting regeneration of mammalian intestinal tissue may refer to regeneration of mammalian intestinal tissue (e.g., atrophied, damaged, aged, dysfunctional, etc.) to at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, of its normal condition as compared to the condition of mammalian intestinal tissue in the absence of contact with an agent or composition described herein.

Aspects of the disclosure involve treating or contacting cells or populations of cells with agents or compositions described herein. As used herein, “treating,” “contacting,” “treating a cell,” “contacting a cell,” “treating a population of cells,” “contacting a population of cells,” and any derivations thereof, are used interchangeably to refer to any means of introducing an agent or composition into sufficient proximity with a target cell for the agent to exert its intended effect on a cell, including chemical and physical means, whether the agent or composition physically contacts the cell directly or is introduced into an environment in which the cell is present. For example, treating or contacting includes binding of an agent to an extracellular or extranuclear domain of a receptor and exerting its effects in that way. Treating or contacting encompass methods of exposing a cell, delivering to a cell, or “loading” a cell with an agent or composition by viral or non-viral vectors, and wherein such agent is bioactive upon delivery. The method of delivery will be chosen for the particular agent and use. Parameters that affect delivery, as is known in the medical art, can include, inter alia, the cell type affected, and cellular location. In some embodiments, treating or contacting includes administering the agent to a subject. In some embodiments, treating or contacting refers to exposing mammalian intestinal tissue (e.g., crypts) or an environment in which the cell line is located (e.g., cell culture) to one or more agents or compositions described herein. In some embodiments, the populations of cells in mammalian intestinal tissue are exposed to an agent or composition in vitro. In some embodiments, populations of cells in mammalian intestinal tissue are exposed to an agent or composition in vivo.

Aspects of the disclosure involve treating a subject with a disease or condition. The terms “treat,” “treatment,” or “treating,” to the extent they relate to a disease or condition, include inhibiting the disease or condition, eliminating the disease or condition, and/or relieving one or more symptoms of the disease or condition. The terms “treat,” “treatment,” or “treating” also refer to both therapeutic treatment and/or prophylactic treatment or preventative measures, wherein the object is to prevent or slow down (lessen) an undesired pathological change or disorder. For example, beneficial or desired clinical results include, but are not limited to, alleviation of symptoms, diminishment of extent of disease or disorder or condition, stabilized (e.g., not worsening) state of disease or disorder or condition, delay or slowing of disease progression, amelioration or palliation of the disease state or disorder or condition, and remission (whether partial or total), whether detectable or undetectable. Those in need of treatment include those already with the disease or disorder or condition, as well as those prone to have the disease or disorder or condition or those in which the disease or disorder or condition is to be prevented. In one embodiment “treat,” “treatment,” or “treating” does not include preventing or prevention.

Aspects of the disclosure involve employing effect amounts of agents and/or compositions. An “effective amount” or “effective dose” of an agent (e.g., TGFB1), or composition containing such agent, generally refers to the amount sufficient to achieve a desired biological and/or pharmacological effect, e.g., when contacted with a cell in vitro or administered to a subject according to a selected administration form, route, and/or schedule. As will be appreciated by those of ordinary skill in the art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. It will be understood that agents, compounds, and compositions herein may be employed in an amount effective to achieve a desired biological and/or therapeutic effect. In some embodiments, an “effective amount” refers to an amount of an agent or composition described herein which is stimulates proliferation, regeneration, and/or self-renewal of intestinal stem cells. In some embodiments, an “effective amount” refers to an amount of an agent or composition described herein which is stimulates proliferation, regeneration, and/or self-renewal of non-stem intestinal progenitor cells. In some embodiments, an “effective amount” refers to an amount of agent or composition described herein which promotes the regeneration of intestinal tissue. The skilled artisan can readily determine the effective amount of an agent or composition described herein for achieving its effective purpose using routine methods, without undue experimentation. It should be understood that in certain embodiments, an effective amount of the agents and compositions disclosed herein may be administered to a subject for a limited period of time (e.g., several days, weeks or months). In certain embodiments, the agents and compositions disclosed herein may be administered to a subject in conjunction with, for example, chemotherapy or radiotherapy, and in such embodiments the agents or compositions may be discontinued upon completion of such chemotherapy or radiotherapy. Similarly, in certain embodiments where such agents or compositions are administered as a pre-treatment, such agents or compositions may be administered prior to initiation of chemotherapy and discontinued upon initiation or completion of the chemotherapy.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure. Unless otherwise defined, all technical and scientific terms used herein have the same meaning as commonly understood by one of ordinary skill in the art to which this invention belongs. In case of conflict, the present specification, including definitions, will control.

The present invention will be described in greater detail by way of specific examples. The following examples are offered for illustrative purposes and are not intended to limit the invention in any manner. Those of skill in the art will readily recognize a variety of noncritical parameters which can be changed or modified to yield essentially the same results.

### EXAMPLES

### Example 1

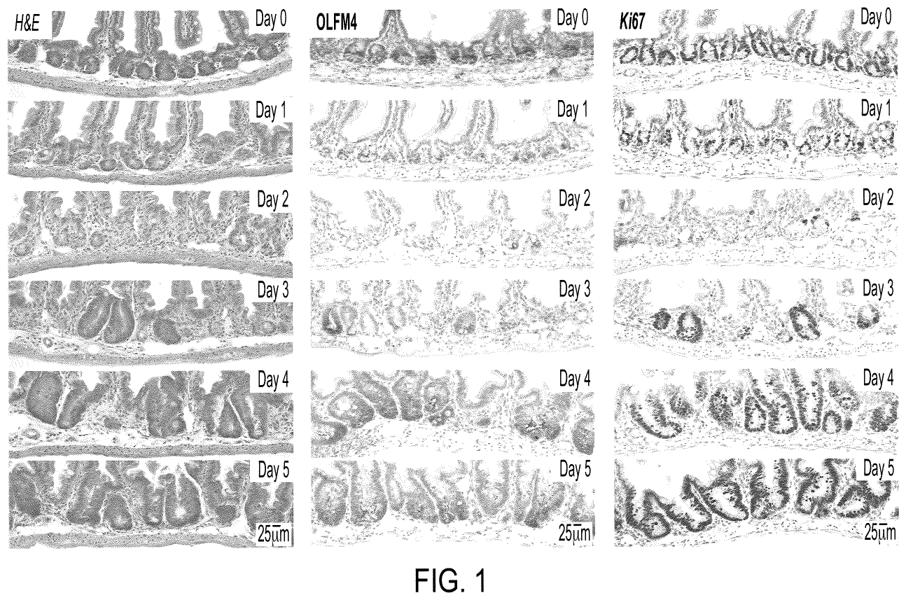

To study the natural healing process in the intestine, mice were subjected to 12 Gy irradiation. A consistent and reproducible pattern was observed in which healthy intestinal crypt cells (where stem and progenitor cells reside in the intestine) were damaged or destroyed within two days following irradiation. However, over the next three days, highly proliferative regenerative clusters of cells expanded and restored the epithelium (FIG. 1). It was hypothesized that by uncovering the mechanisms of this remarkable ability of the native tissue to recover from extensive damage, therapeutic avenues to improve epithelial transplants could be found.

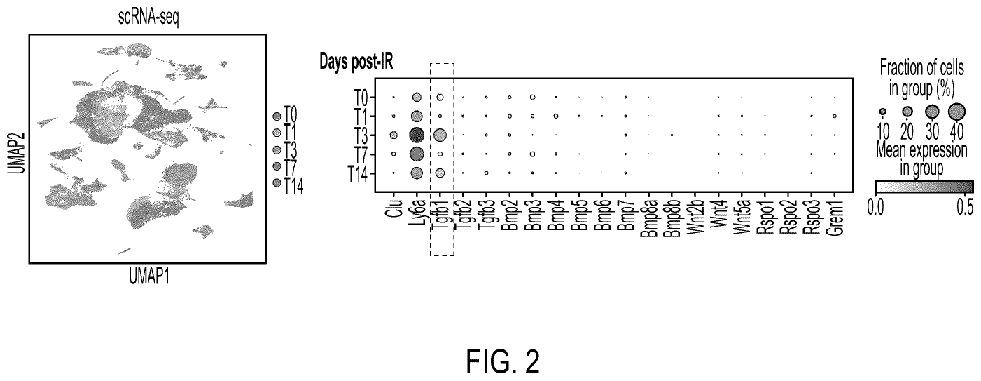

Single-cell RNA sequencing (scRNAseq) data was interrogated in mouse intestinal samples post-irradiation, starting with controls and then 1, 3, 7, and 14 days post-irradiaiton. Factors of known signaling pathways implicated in epithelial cell growth regulation were profiled to identify ligands that exhibit a dynamic pattern of expression, particularly at 3 days post-irradiation when the recovery process begins. TGFB1 expression was notably enriched at this timepoint (FIG. 2), indicating it may be a novel promoter of tissue regeneration.

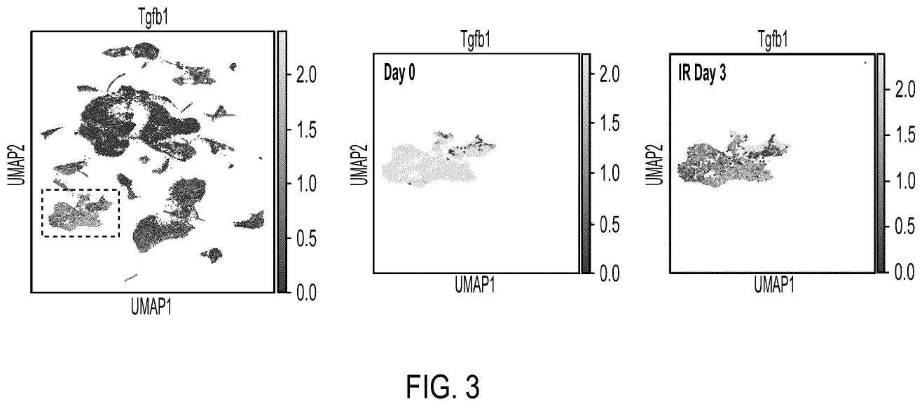

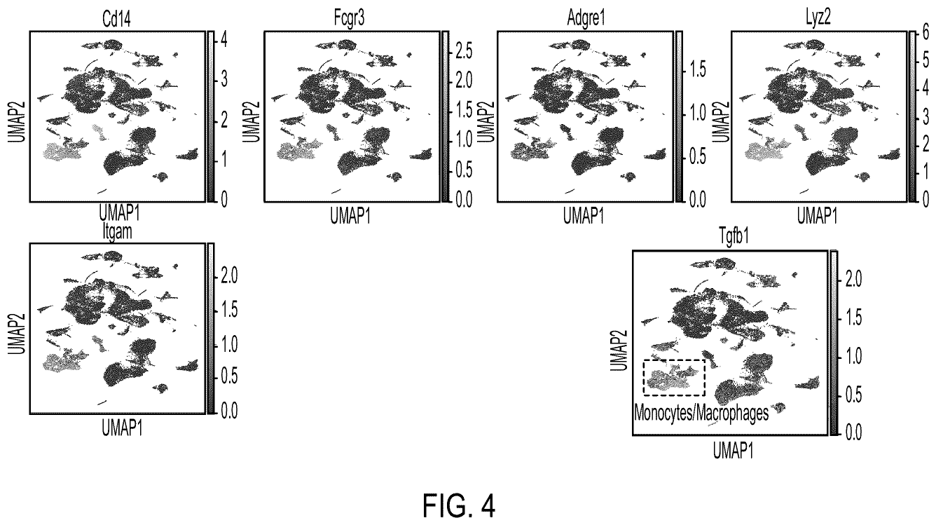

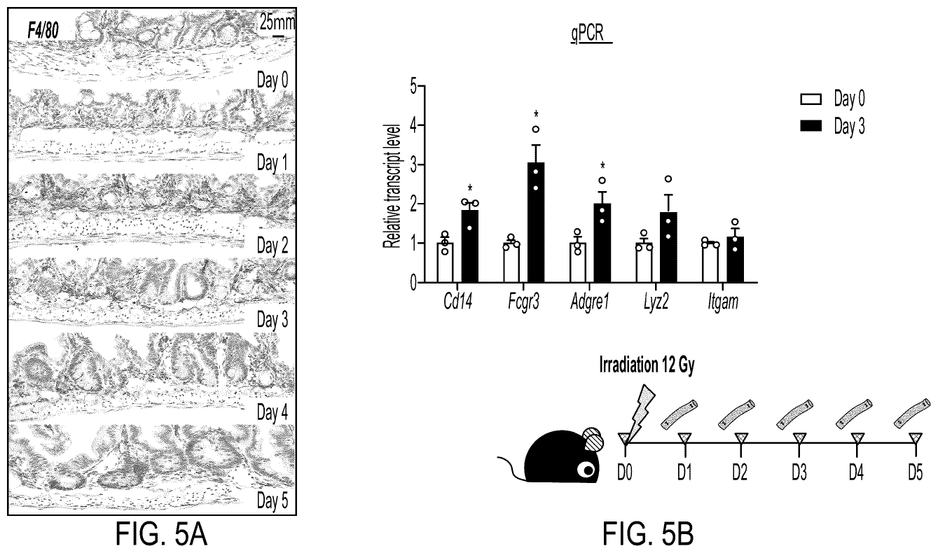

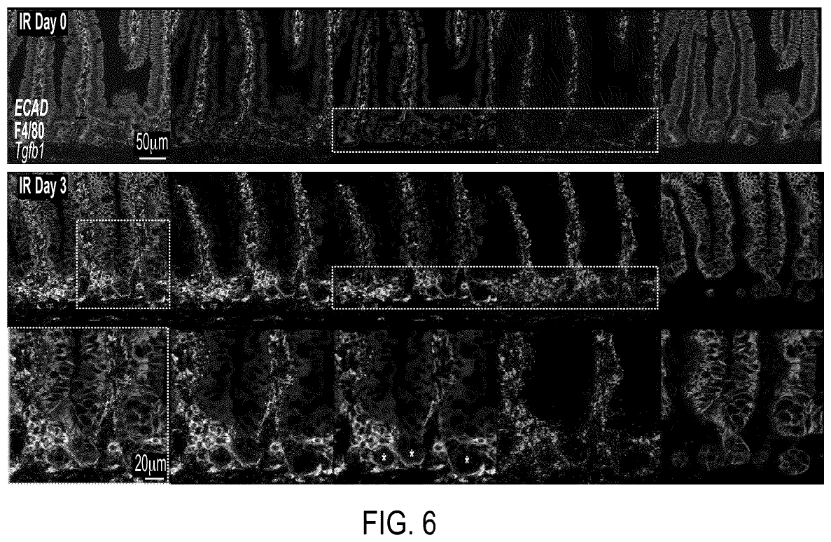

Uniform Manifold Approximation and Projection (UMAP) clustering was then performed to identify cell populations producing Tgfb1. Cells expressing known markers of macrophages/monocytes were the most robust producers of Tgfb1 (FIGS. 3-4). In the tissue samples, macrophage markers were observed to be elevated at 2-3 days post irradiation using immunohistochemistry and PCR (FIGS. 5A-5B). These results were further corroborated by measuring RNA levels in situ using RNAscope, revealing a co-localization between macrophage markers and Tgfb1 expression (FIG. 6).

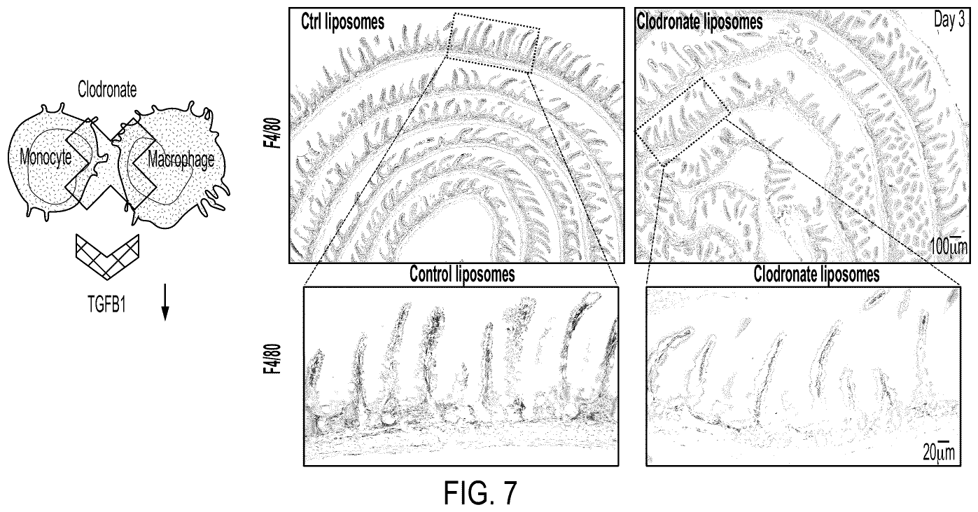

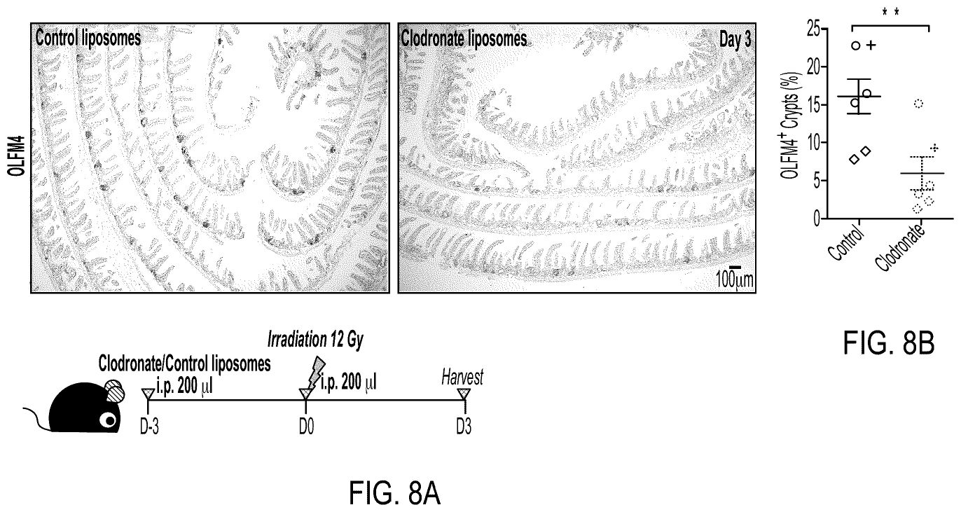

To further evaluate whether macrophage function is important in intestinal regeneration post-irradiation, macrophage cells were depleted from the mice using clodronate-filled liposomes. Depletion of monocyte/macrophage populations was assessed by measuring expression of macrophage markers using Real-Time Quantitative Reverse Transcription PCR (qRT-PCR) (data not shown) and immunohistochemistry on intestinal samples (FIG. 7). A corresponding decrease in Tgfb1 mRNA and protein levels were also observed in the intestines of clodronate-treated mice (data not shown), further suggesting a link between monocyte/macrophages in the intestine and the production of Tgfb1. Importantly, mice with clodronate-depleted macrophage/monocytes showed reduced levels of mRNA epithelial markers of regeneration (not shown), and reduced numbers of regenerative cell clusters in the intestine post-irradiation as marked by OLFM4+ immunostaining (indicating expression of Olfactomedin 4 (OLFM4), a robust marker of epithelial stem cells in the intestine) (FIGS. 8A-8B). Together, these studies indicate that monocytes/macrophages produce Tgfb1 during intestinal regeneration.

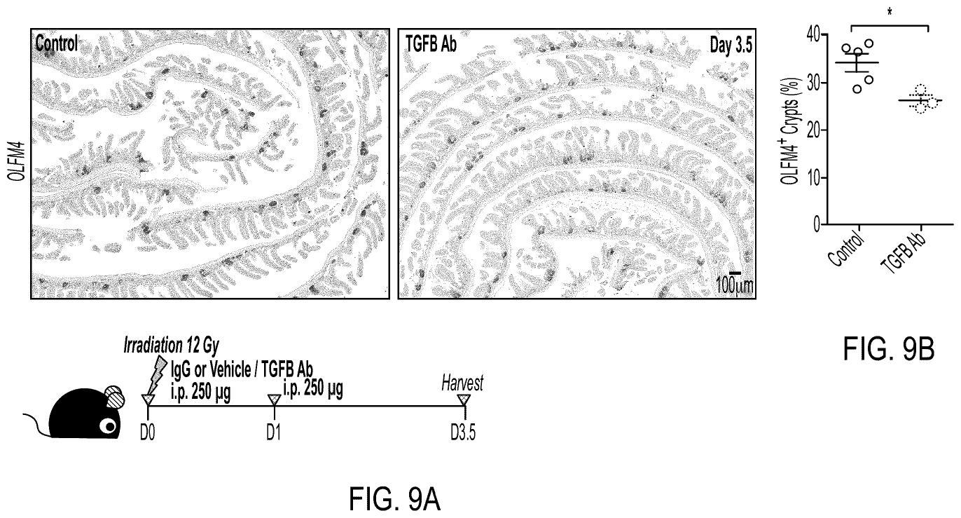

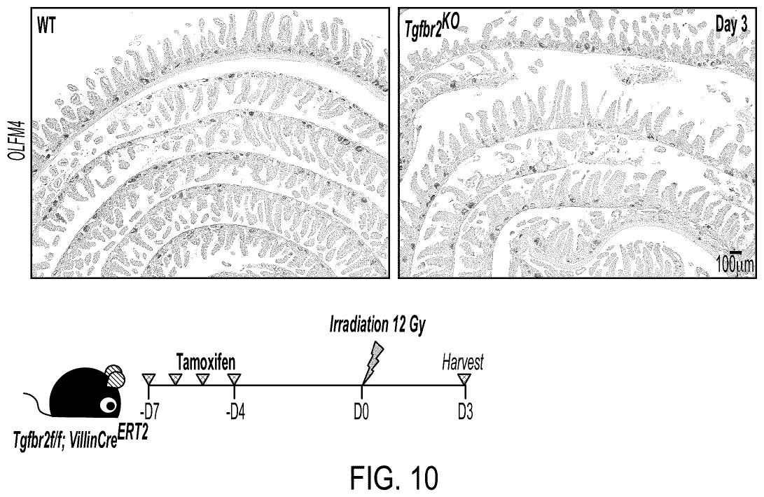

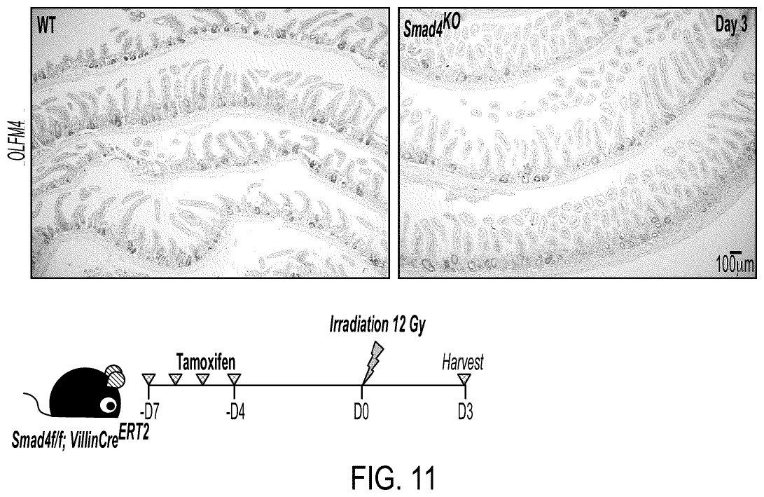

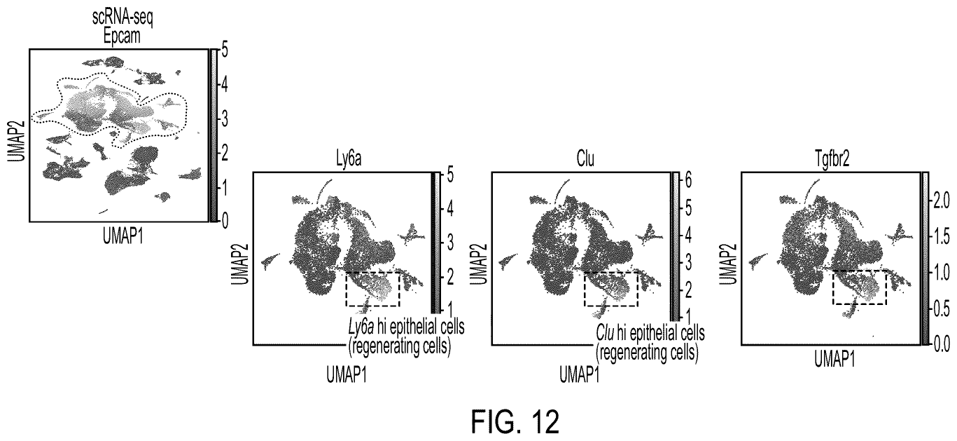

To further test for a role of TGFB1 in intestinal regeneration, mice were either treated with TGFB1-neutralizing antibodies or engineered to lack Tgfbr2, the receptor for TGFB1 ligand. In both cases, disruption of the TGFB1 signaling pathway led to reduced numbers of regenerative foci in the mouse intestines post-irradiation (FIGS. 9A-9B and 10). Additionally, intestine-specific knockout of the downstream transcription factor of TGFB1-signaling, Smad4, yielded a similar reduction of regenerating foci post-irradiation in these genetically altered mice (FIG. 11). Thus, multiple lines of evidence indicate that TGFB1-signaling is required for full intestinal regeneration, and that macrophaged-derived ligand is likely signaling through epithelial receptors to promote intestinal regeneration. In addition, analysis of scRNAseq from the regenerating intestine revealed that epithelial cells, particularly cells in a regenerative state marked by Clu and Ly6a, robustly express the TGFB1 receptor, Tgfbr2 (FIG. 12).

### Example 2

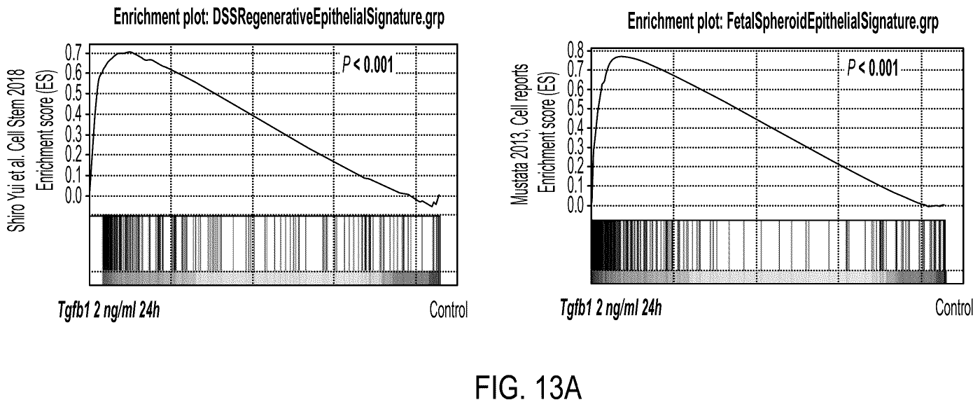

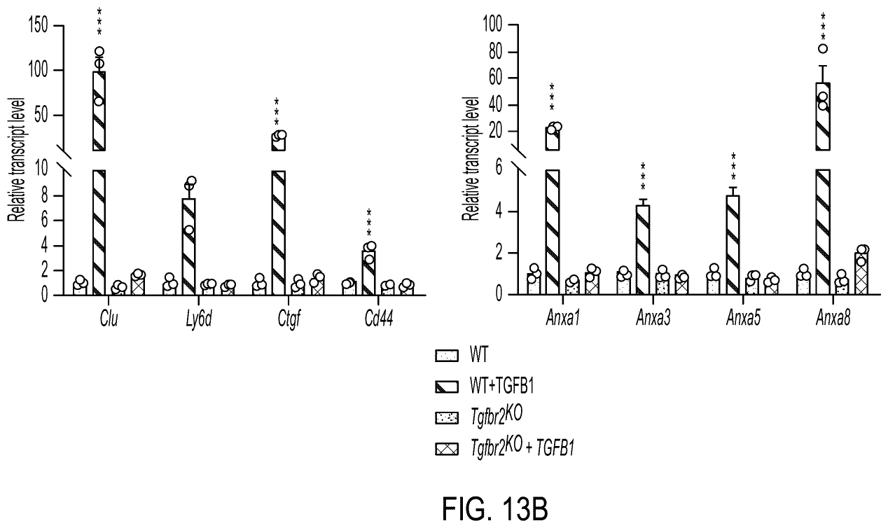

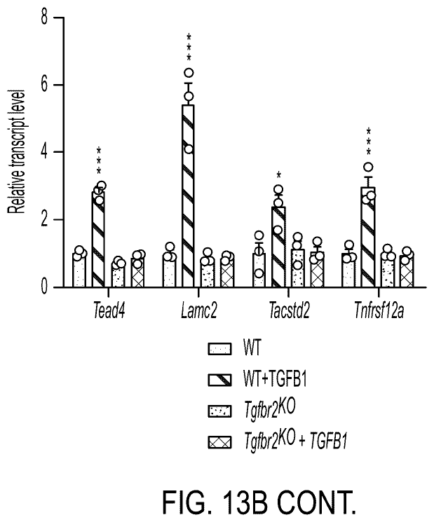

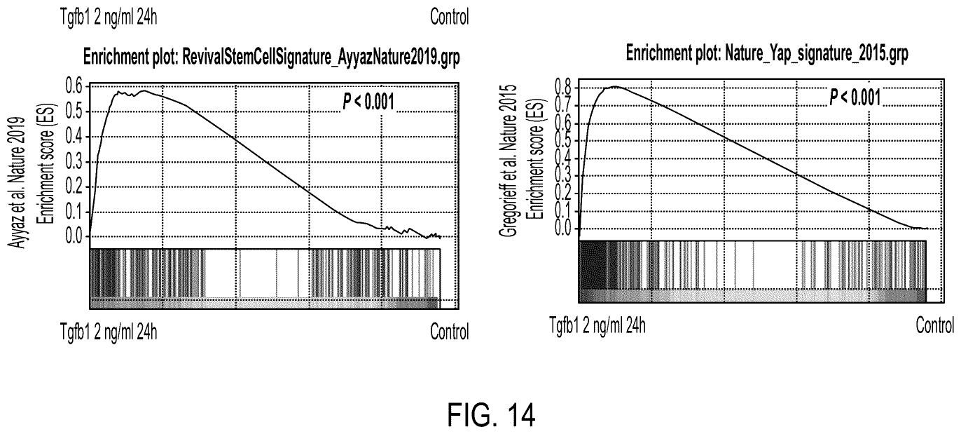

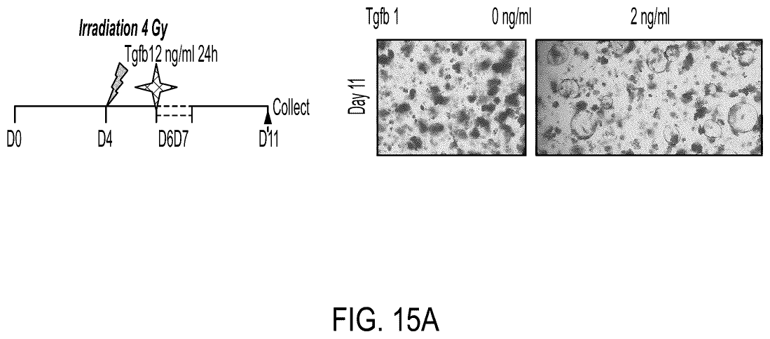

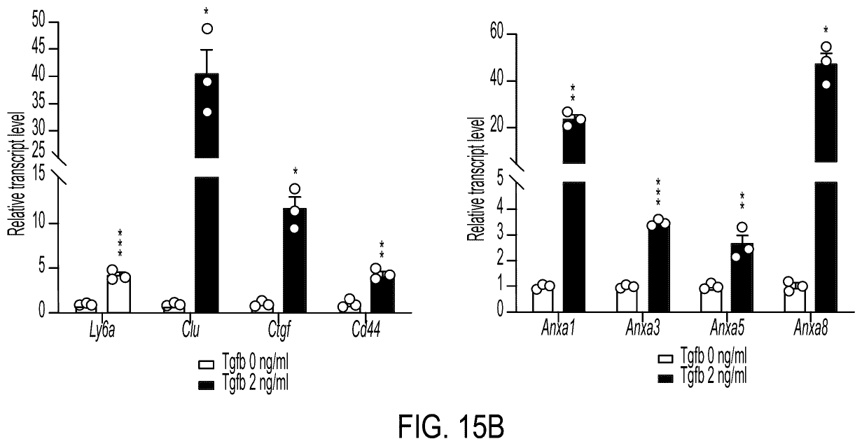

Intestinal epithelial organoid cultures can be derived from primary epithelium and retain cell diversity and gene expression profiles observed in the homeostatic in vivo state (Sato, 2009). To determine whether TGFB1 can trigger a regenerative state in the epithelium, intestinal organoid cultures were treated with TGFB1 and gene expression changes were monitored using RNAseq or qRT-PCR. Remarkably, it was found that organoids treated with TGFB1 underwent a dramatic shift in gene expression profile, upregulating transcripts associated with regenerative epithelium and fetal regengerative epithelium (such as Clu, Ly6, Ctgf, and Tead4) (FIGS. 13A-13B). These changes in expression in response to TGFB1 treatment were dependent upon the organoids expressing the Tgfbr2 receptor, as Tgfbr2 knockout organoids did not show the same response to TGFB1 treatment. In addition, it was found that TGFB1 induces revival stem cell signature YAP gene signaling, which is also important for regeneration (FIG. 14). Morphologically, TGFB1-treated organoids shifted to a spherical appearance reminiscent of fetal-derived organoids, in lieu of the typical, branched morphology of the adult homeostatic state (FIG. 15A). These structures persisted for more than 5 days after a 24-hour TGFB1 treatment, suggesting a sustained response. qRT-PCR analysis showed that these spherical organoids were expressing elevated levels of fetal/regenerative markers such as Clu and Ctgf (FIG. 15B).

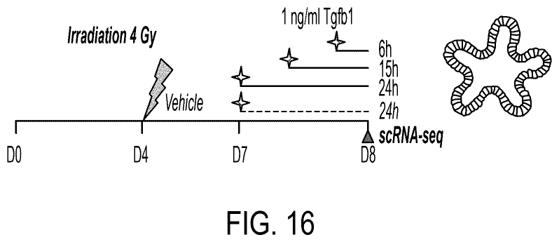

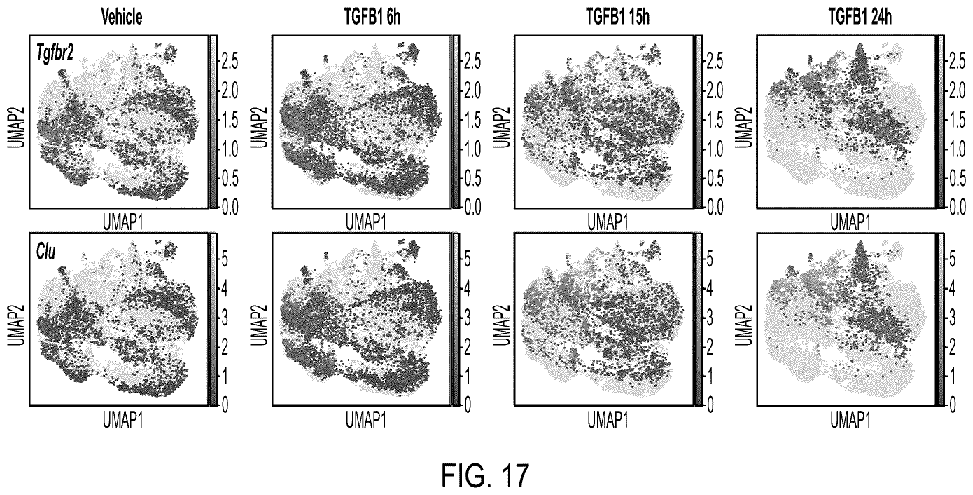

To better understand how organoids were responding to TGFB1-treatment and acquiring a fetal-like/regenerative state, scRNAseq of organoid cultures were performed post-treatment with TGFB1. By 15 hours post-treatment, robust expression of the regenerative marker Clu was induced. This expression pattern overlapped strongly with cells expressing Tgfbr2, suggesting that TGFB1 treatment, working through its receptor, was inducing the regenerative expression program (FIGS. 16-17).

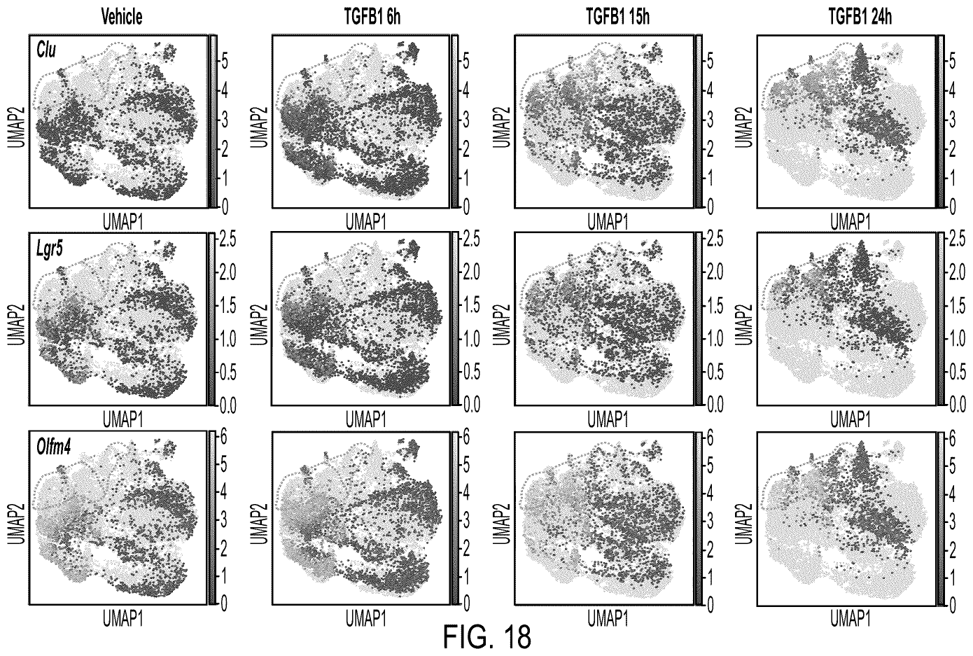

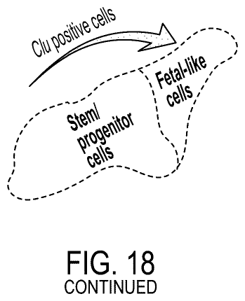

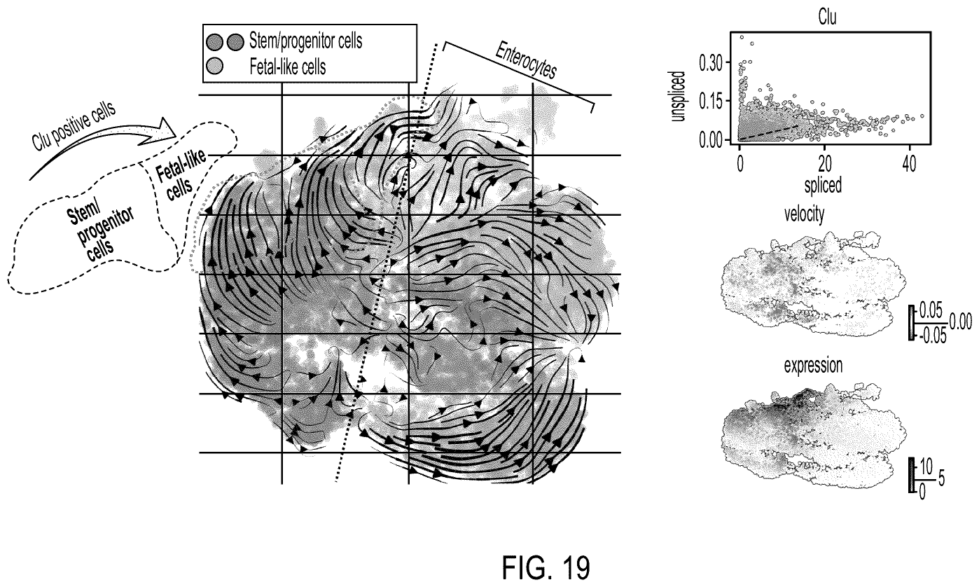

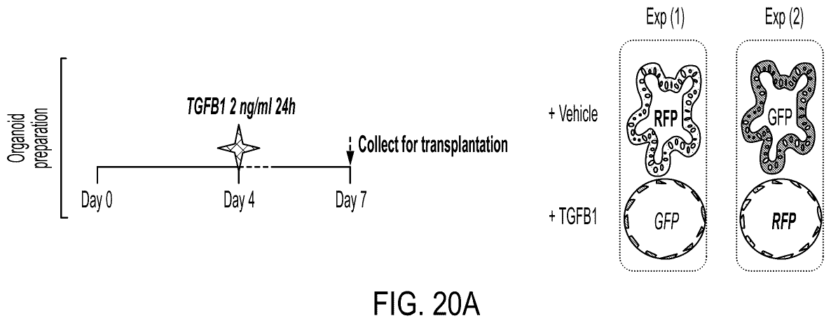

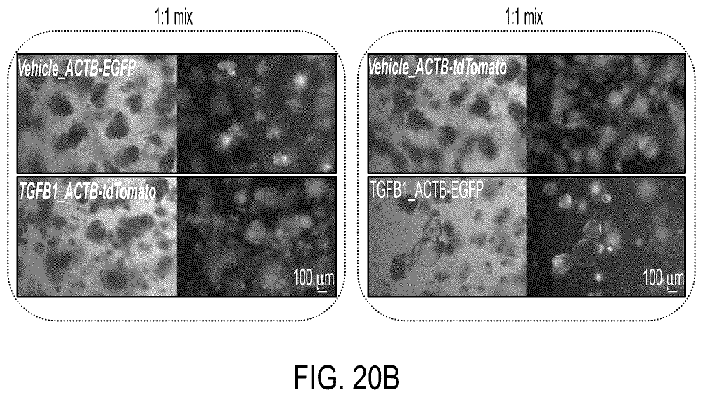

To better appreciate the cells taking on a regenerative phenotype, cell lineage markers of cell clusters adjacent to Clu-expressing cells were examined, and RNA velocity analysis was used to determine which cells were increasing expression of Clu. These analyses suggested that Leucine-rich repeat-containing G-protein coupled receptor 5 (Lgr5)- and Olfm4-expressing cells were likely taking on a regenerative identity in response to TGFB1 treatment. Close proximity was demonstrated between Clu-expressing cell clusters and Lgr5- and Olfm4-expressing clusters (FIG. 18), while RNA velocity analysis suggested that upon TGFB1-treatment, canonical stem cells marked by Lgr5 and Olfm4 undergo a transition to a regenerative state (FIG. 19).

### Example 3

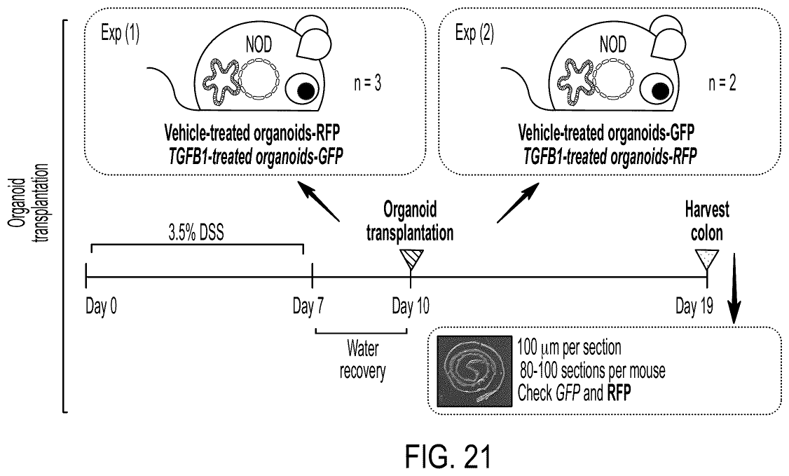

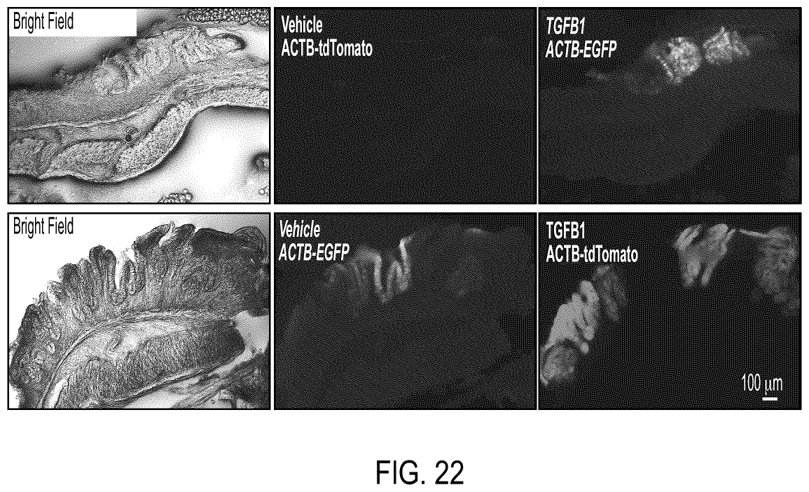

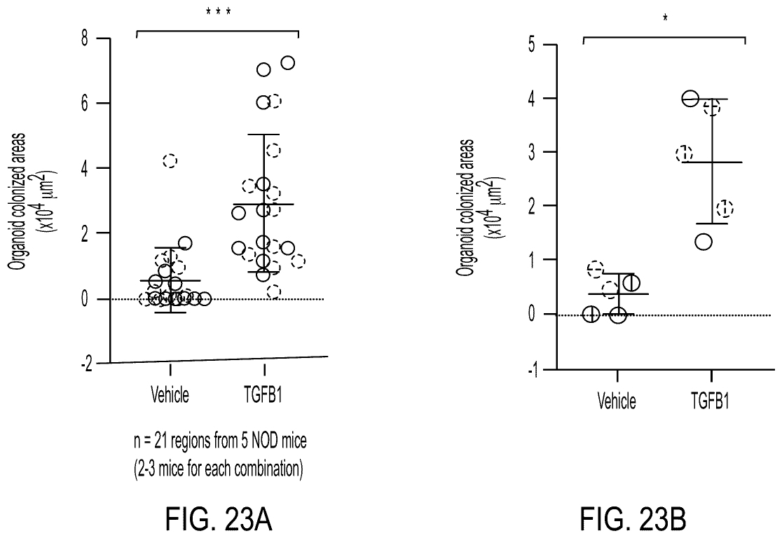

If TGFB1 can induce a regenerative state in organoids, it was hypothesized that TGFB1-treated organoids might produce better engraftment into damaged intestines. A broadly applied mouse model of ulcerative colitis was used to test this hypothesis. See, e.g., Chassaing et al., Dextran Sultfate Sodium (DSS)-Induced Colitis in Mice, Curr Protoc Immunol. 2014 Feb. 4: 104: Unit-15.25.. doi:10.1002/0471142735.im1525s104; Watanabe et al., Transplantation of Intestinal Organoids into a Mouse Model of Colitis, Nat Protoc 17, 649-671 (2022). Immunodeficient mice were treated with dextran sulfate sodium (DSS) to induce an inflammatory state in the distal colon, including ulcerations of the epithelial layer. Assays were then performed to evaluate the ability of control-treated or TGFB1-treated organoids to engraft in the host colon. Organoids from cultures treated canonically or with TGFB1 were transplanted into the mouse colon using a competition assay to increase experimental rigor. Organoids were monitored by expression of transgenic reporters (Red Fluorescent Protein (RFP) or Green Fluorescent Protein (GFP)) and replicate experiments were performed such that in some transplant assays, the RFP organoids were TGFB1-treated, and in others, the GFP organoids were TGFB1-treated. The results of these transplant competition assays demonstrated a clear advantage for TGFB1-treated organoids to engraft in DSS-damaged intestines compared to canonically-treated intestines, with both increased numbers of organoid colonies forming and increased engraftment areas in the recipient colon tissues (FIGS. 21-23).

### Example 4

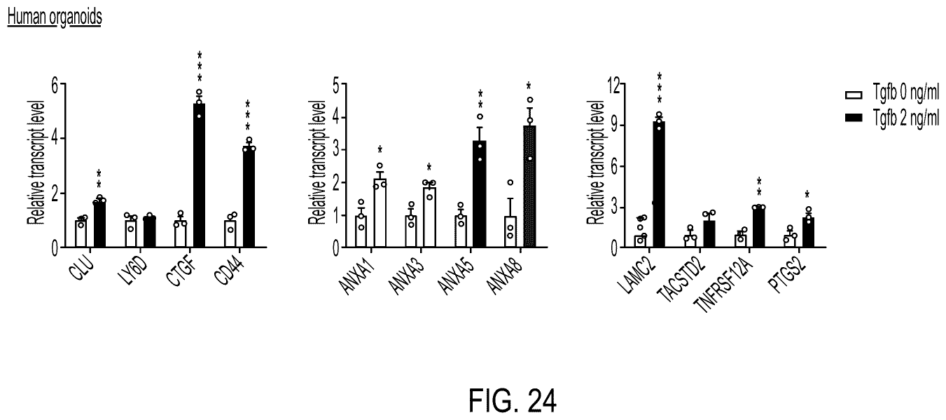

Additional tests were conducted to determine whether human organoids respond similarly to TGFB1-treatment as mouse organoids. Treatment of adult human duodenal organoid cultures with 2 ng/ml TGFB1 induced a similar set of genes observed in the mouse tissue, suggesting that these results can be translated into the human system (FIG. 24). Also see Nakamura et al., Advancing Intestinal Organoid Technology Toward Regenerative Medicine, Cell Mol Gastroenterol Hopatol 5(1): 51-60 (2018).

### Example 5

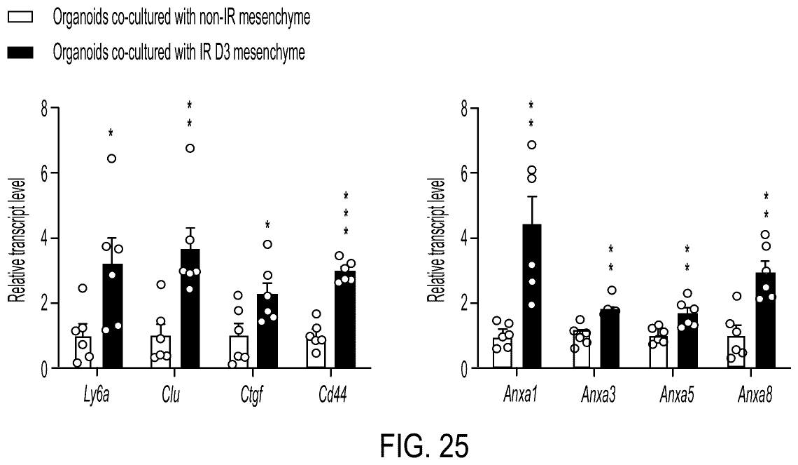

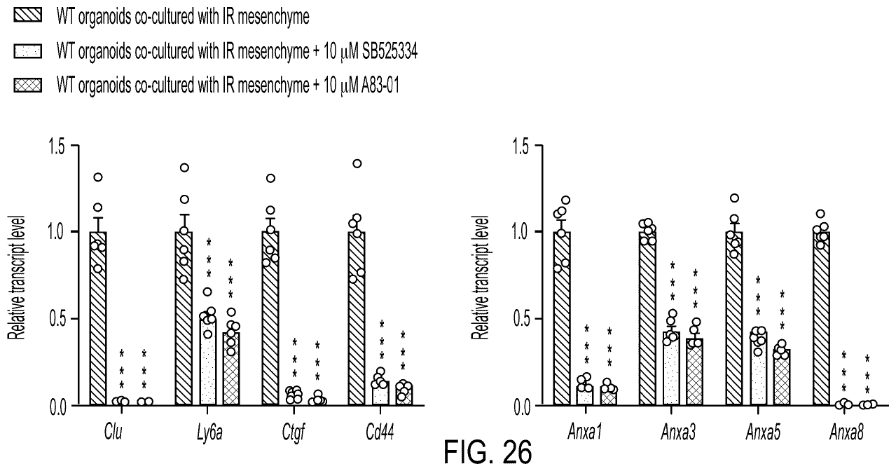

To further explore the relationship between TGFB signaling and the regenerating intestinal epithelium, the ability of intestinal mesenchyme to promote regeneration post-IR was studied. Mesenchyme cells were isolated from non-irradeiated control mice or 12 gy irradiated mice at 3 days post-IR and used in co-culture with intestinal organoids. Mesenchyme from irradiated mice elicited a significant upregulation of regenerative gene expression in the organoids compared to the control mesenchyme (FIG. 25), and this induction of regeneration-marker transcripts appears to be dependent upon TGFB signaling, as co-treatment with TGFB receptor inhibitors severely blunts the induction of these genes by the irradiated mesenchyme (FIG. 26).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of promoting regeneration of one or more intestinal stem cells in a mammalian organoid culture, the method comprising treating the organoid culture with an amount of transforming growth factor beta (TGFB) effective to promote intestinal epithelial regeneration.

2. The method of claim 1, wherein the TGFB comprises transforming growth factor beta-1 (TGFB1).

3. The method of claim 1, wherein the organoid culture comprises cells that express a TGFB1 receptor.

4. The method of claim 3, wherein the TGFB1 receptor is transforming growth factor beta receptor 2 (Tgfbr2).

5. The method of claim 1, wherein the organoid culture comprises epithelial stem cells.

6. The method of claim 1, wherein the TGFB increases stemness of intestinal epithelial cells.

7. The method of claim 1, wherein the organoid culture comprises mesenchymal cells or mesenchymal stem cells.

8. The method of claim 1, wherein the TGFB increases stemness of intestinal mesenchymal cells.

9. The method of claim 1, wherein the organoid culture comprises epithelial regenerative cells

10. The method of claim 9, wherein the epithelial regenerative cells comprise at least one of a Clusterin (Clu) and Stem cell antigen-1 (Ly6a) biomarker.

11. The method of claim 1, wherein the organoid culture comprises a spherical morphology.

12. The method of claim 1, wherein the organoid culture expresses increased levels of fetal or regenerative biomarkers as compared to an untreated control organoid culture.

13. The method of claim 12, wherein the regenerative biomarkers include at least one of Clu and Connective tissue growth factor (Ctgf) biomarkers.

14. The method of claim 1, wherein the organoid culture comprises cells that express at least one of Lgr5 and Olfm4 biomarkers.

15. The method of claim 1, further comprising engrafting the organoid into target intestinal tissue of a subject.

16-28. (canceled)

29. The method of claim 15, wherein the target intestinal tissue comprises an increased number, size, or area of organoid colonies in comparison to intestinal tissue engrafted with an organoid that has not been pre-treated with TGFB.

30-31. (canceled)

32. The method of claim 15, wherein the subject was previously treated with at least one of chemotherapy and radiotherapy.

33. The method of claim 15, wherein the target intestinal tissue is damaged or defective intestinal tissue caused by at least one of the following diseases or disorders: inflammatory bowel disease, graft versus host disease, pathogens and other diseases or disorders causing ulceration of intestinal tissue.

34. The method of claim 15, wherein the target intestinal tissue is damaged or defective intestinal tissue caused by a genetic disease or disorder affecting intestinal epithelial cell functions, the genetic disease or disorder comprising at least one of the following: microvillus inclusion disorders, congenital diarrhea, and nutrient transporter deficiencies.

35. (canceled)

36. The method of claim 34, wherein the organoid comprises a genetically corrected organoid.
